(19) United States
(12) Patent Application Publication
(10) Pub. No.: US 2022/0395637 A1
(43) Pub. Date: Dec. 15, 2022

(54) HYBRID INJECTION-PUMP INSULIN DELIVERY

(71) Applicant: INSULET CORPORATION, Acton, MA (US)

(72) Inventors: Yibin ZHENG, Hartland, WI (US); Joon Bok LEE, Acton, MA (US); Jason O'CONNOR, Acton, MA (US)

(21) Appl. No.: 17/839,645

(22) Filed: Jun. 14, 2022

Related U.S. Application Data
(60) Provisional application No. 63/210,232, filed on Jun. 14, 2021.

(51) Int. Cl. A61M 5/172; A61M 5/142

(52) U.S. Cl. CPC A61M 5/1723; A61M 5/14244; A61M 2230/201

(57) ABSTRACT

The disclosed embodiments are directed to methods for determining, based on input from a manual insulin delivery device, for example, an insulin pen, a proper bolus dosing to be delivered by an AID system, to assess the sufficiency of the user-administered basal dose of long-acting insulin and to recommend any changes to the long-acting delivery following a daily analysis of the user's blood glucose readings.

### RELATED APPLICATIONS

This application claims the benefit of U.S. Provisional Patent Application No. 63/210,232, filed Jun. 14, 2021, the contents of which are incorporated herein by reference in their entirety.

### BACKGROUND

For people with diabetes mellitus, a portion of their daily insulin requirements may be provided by an automatic insulin delivery (AID) system. For example, the diabetic person may manually provide a daily basal dose of insulin at a personalized basal rate, typically via a single injection per day. An AID system may provide automatic bolus does of insulin to compensate for elevated blood glucose levels of the person due to the ingestion of meals and to address any blood glucose excursions experienced by the person. As such, the person may be relieved of the burden of self-administering multiple injections per day.

Typically, it is assumed that approximately 50% of the user's daily insulin requirements are delivered via a single daily basal injection of long-acting insulin, while the remaining 50% is delivered as bolus injections of rapid-acting insulin. In some cases, an AID system may be provided with a sensor capable of providing continuous blood glucose readings from the user. Typical current state of the art AID systems are able to determine, based on the blood glucose readings, the timing and quantity of rapid-acting insulin to be delivered as bolus doses.

### SUMMARY

This Summary is provided to introduce a selection of concepts in a simplified form that are further described below in the Detailed Description. This Summary is not intended to identify key or essential features of the claimed subject matter, nor is it intended as an aid in determining the scope of the claimed subject matter.

Disclosed herein are methods for determining, based on an input of information regarding the quantity of long-acting insulin delivered as a manually-administered basal dose of insulin, the proper bolus dosing to be delivered by an AID system, to assess whether the manually-administered long-acting insulin was sufficient and to recommend any changes to the long-acting basal insulin dose for the next day based on an analysis of the user's blood glucose readings for the current day.

In a first embodiment of the invention, the AID system may be wirelessly connected to an insulin pen such that the quantity of long-acting insulin, as well as the time of delivery of the long-acting insulin, delivered manually by the user via the insulin pen may be automatically reported to the AID system. In this embodiment, when a meal event occurs and the AID system is informed of the meal event either via a notification from the user or via an observed indication of increased blood glucose levels, the AID system can automatically deliver one or more bolus doses of rapid-acting insulin calculated based on the original basal delivery from the insulin pen. Subsequently, the AID system can also suggest changes to the quantity of long-acting basal insulin delivery for the next day based on glucose outcomes.

In one aspect of the first embodiment, a method is provided to suggest bounds to the maximum possible daily basal insulin manually delivered by the user via an insulin pen based on one or more factors, discussed later herein.

In a second embodiment of the invention, the quantity of long-acting insulin delivered by the insulin pen can be utilized to estimate the user's residual insulin needs for delivery by the AID system. In a first aspect of this embodiment, the user's Q:R ratio, which may be used to determine how aggressive the AID system is in compensating for excursions in the blood glucose levels of the user, may be adjusted daily for use in calculations of bolus doses of insulin to be automatically delivered by the AID system during the course of the day. In a second aspect of this embodiment, a new TDI may be adjusted daily for use in calculations of bolus doses of insulin to be automatically delivered by the AID system during the course of the day.

Although exemplary embodiments are described herein as a drug delivery device that administers insulin to a user, the present invention is not limited to the use of insulin. By way of example, other types of drugs that may be administered include (but are not limited to) pramlintide and glucagon-like peptide 1 (GLP-1).

Moreover, some embodiments refer to a bolus dose delivered via an AID in conjunction with a manually-delivered basal dose. However, embodiments are not limited to this configuration, and could be used with (for example) a manually-administered bolus and automatically administered basal dose, or automatically delivered bolus and basal doses.

Preferred embodiments of the invention may comprise the first or second embodiments mentioned above or may comprise a combination of the first and second embodiments mentioned above and any other aspects of the embodiments discussed herein.

### BRIEF DESCRIPTION OF THE DRAWINGS

To easily identify the discussion of any particular element, the most significant digit or digits in a reference number refer to the figure number in which that element is first introduced.

FIG. 1 is a block diagram of an AID system including the basal dosing application of which present invention may be a part.

FIG. 2 is a flowchart showing an overall process for delivering bolus doses for meal compensation, for delivering additional bolus doses to address blood glucose excursions and for recommending adjustments to the user's daily basal dose.

FIG. 3 is a flowchart showing a process for applying a cost function to determine bolus doses delivered by an AID system.

### DETAILED DESCRIPTION

Methods in accordance with the present disclosure will now be described more fully with reference to the accompanying drawings, where one or more embodiments or various aspects of the invention are shown. The methods may be embodied in many different forms and are not to be construed as being limited to the embodiments set forth herein. Instead, these embodiments are provided so the disclosure will be thorough and complete, and will fully convey the scope of the systems and methods to those skilled in the art. Each of the methods disclosed herein provides one or more advantages over conventional systems and methods.

Persons suffering from diabetes may have a portion of their daily insulin requirements delivered via an AID system. As an example, an AID system may provide bolus dosing of rapid-acting insulin to compensate for meal ingestion and blood glucose excursions, while the basal dosing of long-acting insulin may be delivered manually by the person, typically using an insulin pen once a day.

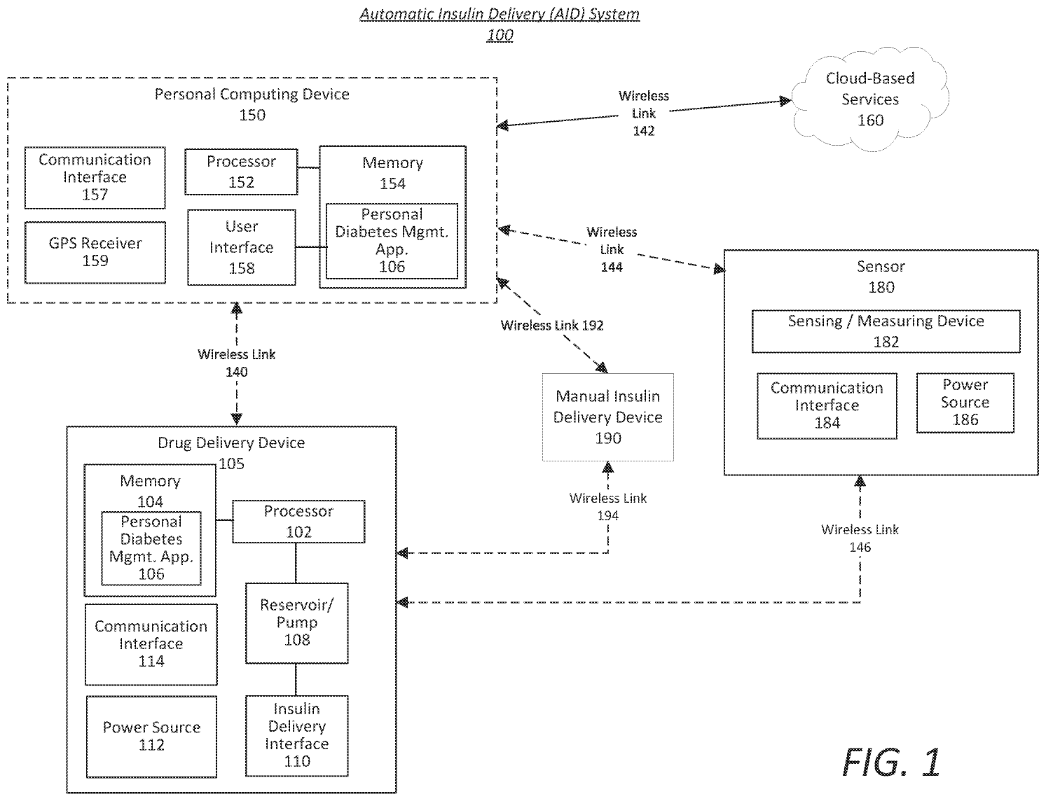

FIG. 1 is a block diagram showing an automatic insulin delivery (AID) system 100 of the type in which the various embodiments of the present invention may be implemented. The AID system 100 includes a personal diabetes management (PDM) application 106 for managing the overall delivery of the user's daily insulin requirements and for controlling the mechanical aspects of a drug delivery device 105 responsible for the automatic delivery of bolus doses of insulin to the user. The PDM application 106 may implement the various novel embodiments of the invention described in the Summary above and in more detail below.

In a primary embodiment of the invention, the PDM application 106 may be implemented as a software application executing on a processor in a drug delivery device 105 as part of AID system 100. Drug delivery device 105 may be, for example, a wearable drug delivery device which is discreetly disposed on the body of the user and held in place on the user's skin by an adhesive. Alternatively, drug delivery device 105 may be, for example, a drug delivery device carried by a user (e.g., on a belt or in a pocket) and having an infusion set with tubing connecting the drug delivery device 105 to a cannula that penetrates the user's skin. In alternative embodiments, PDM application 106 may execute on a personal computing device 150 instead of on drug delivery device 105 and may communicate the bolus dosing requirements to drug delivery device 105 via wireless link 140. In some embodiments, the functionality of PDM application 106 may be split between personal computing device 150 and drug delivery device 105 in any convenient manner. In yet other embodiments of the invention, PDM application 106 may be implemented as a cloud-based service 160 and accessed via wireless link 142 with the user's personal computing device 150.

Drug delivery device 105 may include a processor 102 in communication with memory 104 containing PDM application 106 which is executed by processor 102. When executed by processor 102, PDM application 106 may control a reservoir/pump 108 suitable for delivering insulin to the user via insulin delivery interface 110 which may be, for example, a subcutaneous cannula extending from one or more housings of drug delivery device 105 and into the body of the user. Drug delivery device 105 may further include a wireless communication interface 104. The overall drug delivery device 105 may be powered by power source 112, which may be, for example, batteries or a power harvesting apparatus.

AID system 100 may further include a sensor 180 comprising a sensing/measuring device 182 which may be, for example, a continuous glucose monitor (CGM). Like drug delivery device 105, sensor 180 may also be a wearable device disposed on the body of the user. Sensor 180 may include power source 186 and a wireless communication interface 184.

Drug delivery device 105 and sensor 180 may communicate with each other via wireless link 146. Sensor 180 may provide periodic readings of the blood glucose level of the user to drug delivery device 105 via wireless link 146. For example, sensor 180, in one embodiment, may provide blood glucose level readings to drug delivery device 105 every 5 minutes. Other intervals for reporting the blood glucose levels of the user are within the scope of the invention. In some embodiments of the present invention, the periodic readings of the blood glucose level of the user may be used by PDM application 106, as described below, to determine appropriate bolus doses for meal compensation, to provide additional bolus doses to address blood glucose excursions and to suggest modifications to the user's daily basal dose.

AID system 100 may, in some embodiments, include a manual insulin delivery device 190, for example, an insulin pen, that is capable of connecting wirelessly with either drug delivery device 105 via wireless link 194 and/or with personal computing device 150 via wireless link 192. In embodiments which include the connected manual insulin delivery device 190, the quantities of long-acting insulin manually administered as basal doses by the user may be reported to personal diabetes management at 106 executing on personal computing device 150 or on drug delivery device 105, or having portions executing on personal computing device 150 and portions executing on drug delivery device 105.

In some embodiments, personal computing device 150 may comprise, for example, a smartphone, a tablet device, a smartwatch, or any other personal mobile computing device capable of executing PDM application 106 and communicating with the drug delivery device 105, cloud-based services 160 and sensor 180 via any well-known wireless communication protocol, for example, by forming ad hoc Wi-Fi connections or by connecting via a Bluetooth connection. Personal computing device 150 may be configured with a processor 152 and a memory 154 containing the PDM application 106. Personal computing device 150 may be further configured with a user interface 158 which may be used by the PDM application 106 to enable interaction with a user.

PDM application 106 may have a user interface which may be used, for example, to manually input the user's self-administered basal dosages (in embodiments lacking a connected manual insulin delivery device 190), to allow the user to indicate meal consumption and a macronutrient profile of meals consumed (including, for example, the quantities of carbohydrates, fats and proteins in the meal) and/or to provide feedback to the user regarding the delivery of bolus insulin, suggested basal adjustments and the sensed blood glucose level readings. In some embodiments, the blood glucose level readings may be received from sensor 180, directly via wireless link 144 or indirectly via drug delivery device 105 via wireless link 140.

In alternate embodiments wherein no sensor 180 is used, the user may manually take blood glucose readings and enter the blood glucose readings directly via user interface 158. The blood glucose readings may thereafter be transmitted to drug delivery device 105 via wireless link 140 for use by PDM application 106.

As previously discussed, PDM application 106 may calculate the required bolus doses of insulin, based on an initial assumption that the basal doses which have been self-administered by the user account for no more than 50% of the user's total daily insulin (TDI) requirement. However, under certain circumstances, for example, should the PDM application 106 receive an indication from sensor 180 of an excursion in the user's blood glucose level, additional insulin may be delivered to address the excursion, so as to exceed 50% of the user's TDI.

In preferred embodiments of the invention, PDM application 106 receives periodic blood glucose readings from sensor 180. PDM application 106 may comprise a cycle that is triggered by the receipt of a new blood glucose reading and may, during each cycle, determine if additional bolus doses of insulin are required to address blood glucose excursions as indicated by the current and, optionally, one or more previous blood glucose readings received from sensor 180. In preferred embodiments of the invention, a new blood glucose reading is received from sensor 180 every 5 minutes.

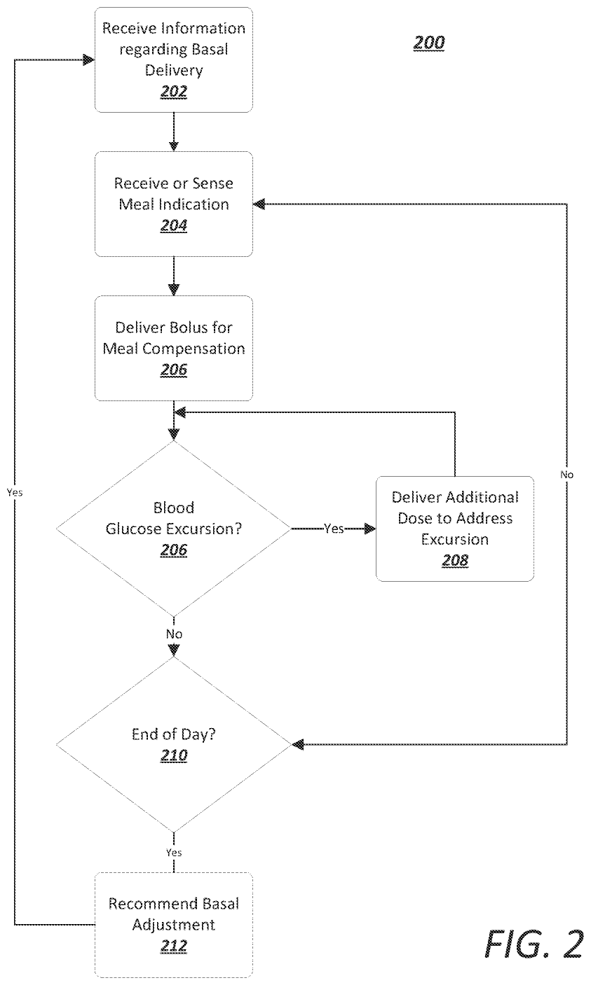

In a first major embodiment of the invention, AID system 100, using PDM application 106, provides bolus doses of rapid-acting insulin, delivered via drug delivery device 105, to compensate for ingested meals and to address excursions in the user's blood glucose levels. A process 200 comprising the first embodiment of the invention is shown in flowchart form in FIG. 2.

At 202, the AID system 100 receives information regarding the delivery of the basal insulin. In one aspect of the invention, as previously discussed, AID system 100 may include a connected manual insulin delivery device 190 which is capable of informing the PDM application 106 of the quantity of long-acting insulin delivered as a basal dose. Manual insulin delivery device 190 may comprise an insulin pen used by the user to manually administer the dose of basal insulin, typically on a once per day basis. Preferably, the manual insulin delivery device 190 will have the capability to form a wireless connection with personal computing device 150 and/or drug delivery device 105, either of which may be executing all or a portion of PDM application 106. In a second aspect of the invention, the user may use a non-connected manual insulin delivery device 190 which may be, for example, a non-wireless-enabled insulin pen or a syringe. In this aspect of the invention, the user must indicate to AID system 100 the quantity of long-acting insulin delivered as a basal dose via the user interface of PDM application 106 running on personal computing device 150.

At step 204, PDM application 106 determines the occurrence of a meal event. In a first aspect of the invention, the user informs the AID system 100 that a meal event is occurring or will occur via the user interface of the PDM application 106 running on personal computing device 150. In this aspect of the invention, the user may also provide, in addition to the notification, a macronutrient profile of the meal which comprises, at least, the quantity of carbohydrates contained in the meal. The macronutrient profile of the meal may also include additional information, for example, the quantity of fat and protein in the meal and/or the ratios of carbohydrates to fat and carbohydrates to protein in the meal, or an indication of the foods in the meal. In this aspect of the invention, PDM application 106 determines an appropriate bolus dose to address the expected blood glucose levels based on the information regarding the macronutrient profile of the meal.

In a second aspect of the invention, AID system 100 may automatically detect the meal event by observing a rise in blood glucose levels as indicated by blood glucose readings from sensor 180. In this case, the increase in the blood glucose level is treated as a blood glucose excursion and an appropriate bolus dose is calculated accordingly.

At step 206, once the appropriate dose of rapid-acting insulin is determined, PDM application 106 may control a reservoir/pump 108 on drug delivery device 105 to deliver an appropriate bolus dose to the user via insulin delivery interface 110.

At steps 206 and 208 of process 200, the method determines if a blood glucose excursion is occurring and may deliver additional bolus doses of rapid-acting insulin above and beyond the insulin required to compensate for the meal event. In one embodiment, an excursion is determined to have occurred once the algorithm experiences a predetermined number of cycles wherein the received blood glucose reading exceeds a high threshold. In one embodiment of the invention, the high threshold may be, for example, 300 mg/dL. In other embodiments of the invention, other thresholds may be used. In this case, a cycle is triggered each time personal diabetes manager application 106 receives a new blood glucose reading from sensor 180. In preferred embodiments of the invention, a cycle will therefore be triggered every five minutes; however, periodic readings from the blood glucose sensor 180 may be received at any predetermined periodic interval.

Examples of actions taken to address blood glucose excursions will now be provided, assuming five-minute cycles. In one example, at step 206, when PDM application 106 has received 12 consecutive blood glucose readings above the high threshold, indicating that the user's blood glucose levels have been elevated for approximately one hour, PDM application 106 may determine that a delivery of an additional bolus dose of rapid-acting insulin is required. The quantity of rapid-acting insulin delivered in the additional bolus may be determined by Eq. (1) and delivered at step 208 of process 200:

$${I}_{bolus} = 0.05 ( T D I ) - IOB ( i ) + \frac{B {G}_{CGM} ( i ) - B {G}_{target} ( i )}{C F} \qquad ( 1 )$$

where:
TDI is the user's total daily insulin;
IOB(i) is the insulin on board, or, the total remaining insulin in the user's body for the current cycle i;
BG_{CGM}(i) is the blood glucose level reported by the sensor for cycle i;
BG_{target}(i) is the target blood glucose level for cycle i; and
CF is a correction factor, also referred to as “insulin sensitivity”. CF specifies how much BG can be lowered by one unit of insulin (U). The higher the correction factor, the less insulin is required to lower a set amount of BG.

Note that the insulin on board may be calculated based on known rates of in-body decay of both long-acting insulin and rapid-acting insulin. For example, the rate of decay of the long-acting insulin in the body is much smaller than the rate of decay of the rapid-acting insulin in the body. Therefore, a total insulin on board can be estimated as the total long-acting insulin left in the body from the user's daily basal dose, based on the rate of decay for long-acting insulin, plus any remaining rapid-acting insulin in the body from previous bolus doses provided by AID system 100, based on the rate of decay for rapid-acting insulin. The rate of decay may be calculated based on a time of injection of the long-action insulin received from the insulin pen.

In this aspect of the invention, again at step 206, in the event that the user's blood glucose level remains above the high threshold for an additional 12 cycles, indicating that the user's blood glucose has remained elevated for over two hours (2*5 minutes*12 cycles), at 208, an additional bolus dose may be administered in accordance with Eq. (1). If, at 206, the user's blood glucose levels are still above the high threshold after an additional 3 hours, one or more additional bolus doses may be administered at 208 in accordance with Eq. (1).

As would be realized by one of ordinary skill in the art, the criteria used to determine whether additional bolus doses are required (i.e. whether a blood glucose excursion has occurred), the exact timing of the delivery of the additional bolus doses, as well as the equation used to calculate the quantity of rapid-acting insulin comprising the additional bolus doses may be different in different implementations of the invention and are provided as above only in an exemplary capacity. The invention is not meant to be limited to these specific examples.

At 210, process 200 determines if the end of the day has occurred and if not, returns to step 204 awaiting the indication of an additional meal ingestion by the user. In various embodiments, the end of the day could be midnight or could be the end time of any contiguous 24-hour period, which could be a user-settable option. If the end of the day has occurred, control proceeds to step 212 where the basal dose delivered by the user may optionally be recommended for adjustment.

To make the recommendation to increase or decrease the user's manually-administered basal dose, the assumption is made that, at most, 50% of the user's TDI is addressed by the basal dose of long-acting insulin. Continuing the example above, if the user's blood glucose readings indicate blood glucose levels above the high threshold for 12 consecutive cycles, indicating an elevated blood glucose level for a period of at least an hour, PDM application 106 may recommend that the user increase the basal dose for the next day. In one embodiment, the increase in the user's manually-administered basal dose is a percentage of the user's TDI. In one embodiment, the adjustment could be 5% of the user's TDI. If the user's blood glucose levels continue to be above the high threshold for an additional 12 cycles, for a two hour time period during which the user's blood glucose levels are elevated, the recommendation for an increase in the user's basal dose may be increased by an additional 5% to 10% of the user's TDI.

In addition, PDM application 106 may recommend a decrease in the user's basal dose. For example, in the event that the user's blood glucose level falls below a low threshold for an extended time period, for example, 6 consecutive cycles during the course of a day, PDM application 106 may recommend that the basal dose be reduced by 5% of the user's TDI. Should the blood glucose readings indicate a blood glucose level below the low threshold for an additional 6 consecutive cycles, PDM application 106 may recommend a decrease in the basal dosage by an additional 5%, to 10% of the user's TDI. Note that, in some embodiments of the invention, the low threshold may be, for example, 54 mg/dL; however, this value is exemplary only and any blood glucose reading could be used as a low threshold (e.g., 60 mg/dL or 70 mg/dL).

As would be realized by one of ordinary skill in the art, the criteria for determining whether a recommendation should be made to adjust the basal dosage provided above are provided only in an exemplary capacity. The actual criteria for determining whether the recommendation should be made, as well as the actual recommendation, may vary in other implementations of the invention without deviating from the scope of the invention.

In the event that the blood glucose readings indicate periods of both hyperglycemia and hypoglycemia during the course of a day, PDM application 106 preferably prioritizes a hypoglycemic risk as the AID system 100 can always deliver additional insulin to compensate for hyperglycemia, but cannot remove excess insulin causing the hypoglycemia from the user's body once it has been administered.

In certain embodiments of the invention, at the beginning of the day, prior to the user manually administering the basal dose, the user may be provided with a suggested maximum insulin delivery amount that varies each day, based on various factors.

In this embodiment, the first factor may be, for example, the total insulin delivery during the previous day. The system would first estimate the overall insulin delivery by the system, including both the long-acting insulin delivered manually by the user and the automated insulin delivery delivered by drug delivery device 105. It is desirable that no more than approximately 50% of insulin needs for the day should be covered by the user-administered basal dose. This factor, F₁, may therefore be calculated utilizing Eq. (2):

where:

$${F}_{1} = 0.5 {\sum }_{j = i - 288}^{i} I ( j ) \qquad ( 2 )$$

l(j) is the total insulin delivered during any cycle i during the previous day. Note that there are 288 five-minute cycles which occur each day, and that the constant in Eq. (2) may therefore vary based on the length of the cycles. A cycle may be determined by the periodicity of the delivery of the blood glucose readings from sensor 180.

In this embodiment, a second factor may be the average insulin needs over time. PDM application 106 may review any trends in the user's insulin needs over time, as the trends may indicate a change the user's diabetes prognosis, and thus a reduction (or increase) in the user's insulin needs may be indicated. This factor, F₂, may be calculated using Eq. (3):

$${F}_{2} = 0.5 ( \frac{{Σ}_{j = i - 288}^{i} I ( j ) + {Σ}_{j = i - 288 \cdot K}^{i - 288 \cdot ( K - 1 )} I ( j )}{2} ) \qquad ( 3 )$$

where:
K is a tunable parameter.

Eq. (3) takes a simple average of the user's TDI K days ago versus the TDI of the current day. Alternatively, the trend in insulin delivery each day may be incorporated into a predicted rate of change based on Eq. (4):

$${F}_{2} = 0.5 {\sum }_{j = i - 288}^{i} I ( j ) + T D {I}_{R o C} \qquad ( 4 )$$

$$where : T D {I}_{R o C} = {\sum }_{j = i - 288}^{i} I ( j ) - {\sum }_{j = i - 288}^{i} I ( j )$$

In this embodiment, a third factor can be, for example, the average expected variation in the user's insulin needs. It is known that the user's insulin needs can vary significantly due to intra-personal variations, such as sickness, dawn phenomena, activity level, lifestyle changes, and diet choices. As an initial estimate, this third factor, F₃, can be estimated in accordance with Eq. (5) based on the standard deviation of the TDI in the previous three to five days:

$${F}_{3} = {\sum }_{j = i - 288}^{i} I ( j ) + {σ}_{T D {I}_{n} ∼ T D {I}_{n - 2}} \qquad ( 5 )$$

In this embodiment, yet another factor can be, for example, based on the average maximum insulin compensation that can be delivered by the AID system 100. This factor can assess, in addition to the automated insulin delivery during the previous day, how much additional insulin AID system 100 may potentially deliver, given various constraints placed on AID system 100. For example, drug delivery device 105 may be constrained, for the safety of the user, to non-CGM dependent constraints. As an example, one non-CGM dependent constraint may limit the amount of insulin that the user would receive within a short period of time to three times the user's TDI-based basal rate. The non-CGM dependent constraints may vary in various implementations of PDM application 106. This factor, F₄, may be calculated using equation 6:

$${F}_{4} = {\sum }_{j = i - 288}^{i} {I}_{manual} ( j ) - \frac{9}{24} {\sum }_{j = i - 288}^{i} I ( J ) \qquad ( 6 )$$

The recommended maximum daily injection can thereafter be calculated as an average of the four previously discussed factors in accordance with Eq. (7):

$${I}_{manual , max} = \frac{{F}_{1} + {F}_{2} + {F}_{3} + {F}_{4}}{4} \qquad ( 7 )$$

Note that Eq. (7) provides for an equal weighting of each factor. In variations of this embodiment, the weighting of each factor can be varied based on the relative importance or accuracy that can be provided by each factor, and a weighted average can be provided as a recommendation to the user. In various embodiments of the invention, any combination of the four factors may be used in a weighted or non-weighted average, or in any other way to determine the maximum basal dose to recommend to the user.

In a second major embodiment of the invention, the quantity of long-acting insulin delivered as a basal dose by the user can be utilized to estimate the user's residual insulin needs for automated insulin delivery by AID system 100. Specifically, the portion of the user's TDI that has been manually administered by the user can be assessed as a factor within the calculation of the user's Q:R ratio.

To calculate a bolus dose of insulin, a cost function is applied to one or more possible dosage options and the dosage option having the best value of the cost function may be selected for delivery to the user. The cost function has a glucose cost component and an insulin cost component. The glucose cost represents the difference between the projected trajectory of the user's blood glucose level over an interval should the adjusted amount be chosen for delivery given the current blood glucose level and the target blood glucose level. The glucose cost penalizes positive blood glucose level excursions from the target blood glucose level. The insulin cost represents the net difference between a deviation of the delivery dosage option from the current basal insulin dosage and a converted amount of insulin needed to compensate for glucose excursions above a target for an interval of time. The cost function is explained in detail in pending U.S. patent application Ser. No. 17/330,115, filed May 25, 2021, the contents of which are incorporated herein in their entirety.

The cost function has a glucose cost weight coefficient (Q) for weighting the glucose cost component and an insulin cost weight coefficient (R) for weighting the insulin cost component. The Q:R ratio is a key parameter for determining the aggressiveness of providing additional bolus doses of insulin to address blood glucose excursions such that the blood glucose level excursions will be weighted more heavily than insulin excursions. The weight coefficients Q and R conventionally are fixed based on clinical parameters for the user. Thus, the insulin delivery of AID system 100 will not vary for a fixed set of clinical parameters for the user. As a result, the clinical parameters must change for the AID system 100 to improve the control performance at a given blood glucose level. A new value for the glucose cost weight coefficient, Q, may be derived using Eq. 8:

$${Q}_{n e w} = {Q}_{b a s e} \cdot P \qquad ( 8 )$$

$$where : P = max ( \sqrt{\frac{{I}_{pen}}{0.5 \cdot T D I}} , 0.25 )$$

where:
lₚₑₙ is the quantity of long-acting insulin in the basal dose manually administered by the user for the current day.

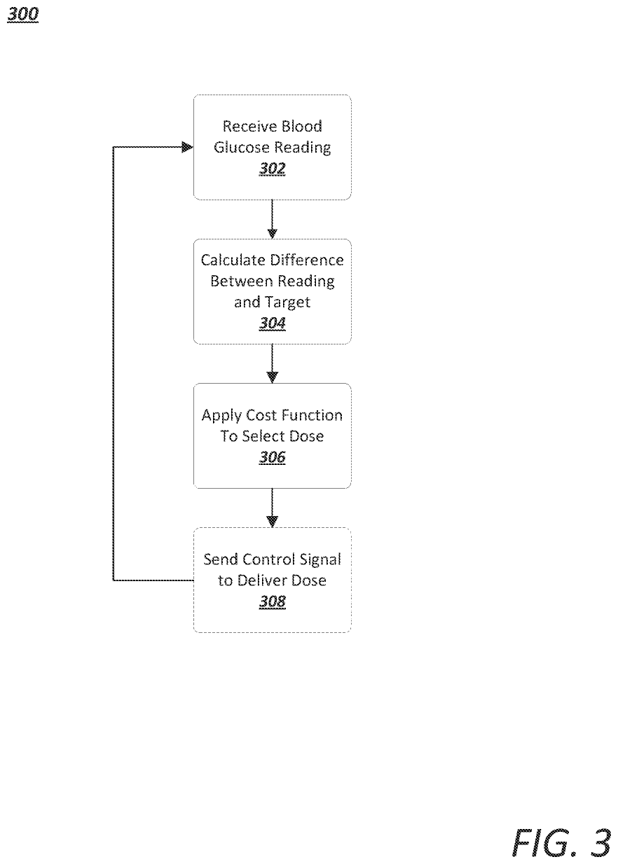

The new Q coefficient can be recalculated daily to determine the Q:R ratio to be used to calculate bolus doses for the user for that day. The process 300 for determining the dosage of rapid-acting insulin to be delivered by AID system 100 is shown in FIG. 3. At 302, the blood glucose reading is received by PDM application 106 from sensor 180. At 304, the blood glucose reading is used to determine the difference between the current blood glucose level of the user and a target blood glucose level for the user. This calculation may occur each cycle or may be averaged over a predetermined number of previous cycles. At 306, based on the difference between the reading and the target, several possible doses may be selected for delivery to the user and the cost function may be applied to the several possible doses. The dose having the best value of the cost function (i.e., the lowest cost) is selected for delivery to the user. At 308, a control signal may be sent from PDM application 106 to reservoir/pump 108 to initiate the delivery of the insulin to the user via insulin delivery interface 110. Note that each time a new dose is determined, the dose may be delivered immediately, or various doses may be aggregated over several cycles and delivered as a single dose.

Alternatively, the user's TDI, which has been either input manually via PDM application 106 or has been calculated based on information from a connected manual insulin delivery device 190 regarding the quality of the long-acting insulin in the manually-delivered basal dose, can be similarly directly adjusted for use in determining the user's residual insulin needs based on Eq. (9):

$$T D {I}_{n e w} = T D I \cdot \frac{P}{0.9} \qquad ( 9 )$$

The new value for the user's TDI may be used, for example, in Eq. (1) as a TDI value for calculation of a bolus dose of insulin to be delivered by AID system 100.

In various implementations of the invention, any embodiments or aspects of the invention discussed herein may be used alone or in any combination. As would be realized, specific equations and thresholds used to determine the quantity and timing of insulin to be delivered by AID system 100 are provided in an exemplary context only and are not meant to limit the scope of the invention. Likewise, any constants appearing in any of the aforementioned equations should be considered tunable parameters which may be modified based on specific implementations of the invention.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A method comprising:
receiving a quantity of a basal dose of insulin;
determining, during the course of a day, that a blood glucose excursion has occurred based on one or more blood glucose readings from a continuous blood glucose monitor;
calculating a bolus dose of insulin to address the blood glucose excursion; and
based on the blood glucose excursion, recommending an adjustment to the quantity of the manually-administered basal dose for a subsequent day.

2. The method of claim 1, wherein:
the bolus dose is administered by an automated insulin delivery (AID) device and the basal dose is administered by a manual insulin pen, or
the basal dose is administered by the AID device and the bolus dose is administered by the manual insulin pen.

3. The method of claim 1 further comprising informing a user of the recommended adjustment prior to administration of the basal dose for the subsequent day on a display device.

4. The method of claim 1 wherein a blood glucose excursion is determined to have occurred after receiving a predetermined number of blood glucose readings above a high threshold or a predetermined number of blood glucose readings below a low threshold.

5. The method of claim 4 wherein the blood glucose readings are received from a wirelessly connected continuous glucose monitor.

6. The method of claim 1 wherein the quantity of the basal dose of insulin is received from a wirelessly connected insulin pen.

7. The method of claim 1 wherein the bolus dose of insulin is calculated based on a total daily insulin and insulin on board and the quantity of the basal dose is assumed to be no greater than 50% of a total daily insulin requirement.

8. The method of claim 7 wherein the insulin on board is a sum of long-acting insulin administered in the basal dose adjusted for a time since administration of the basal dose based on a decay rate for long-acting insulin and rapid-acting insulin delivered as bolus doses adjusted for a time since administration of each bolus dose based on a decay rate for rapid-acting insulin.

9. The method of claim 1 wherein the adjustment to the manually-administered basal dose for the next day is based on the number of blood glucose readings exceeding the high threshold or low threshold.

10. The method of claim 9 wherein the adjustment is a percentage of a total daily insulin requirement.

11. The method of claim 1, further comprising calculating a maximum adjustment to a basal dose of insulin, the calculating comprising:
averaging one or more factors selected from a group consisting of a total insulin quantity of delivery in a previous day, an average insulin need of a user over time, an average expected variation in the insulin need of the user, and an average maximum insulin compensation that can be provided by an AID system.

12. The method of claim 11 wherein each of the factors are weighted such that the maximum adjustment is a weighted average.

13. The method of claim 11 wherein the maximum adjustment is applied to the recommended adjustment to the manually-administered basal dose for the subsequent day of claim 1.

14. A method for estimating a residual insulin need for delivery by an automatic insulin delivery system comprising:
calculating an adjustment factor as a function of a manually-administered basal dose of insulin.

15. The method of claim 14 wherein the adjustment factor is used to calculate a new Q:R ratio for use in calculating bolus doses of insulin to be delivered by the automatic insulin delivery system.

16. The method of claim 14 wherein the adjustment factor is used to calculate an adjusted total daily insulin quantity for a user, the adjusted total daily insulin quantity being used to calculate bolus doses of insulin administered by the automatic insulin delivery system to compensate for blood glucose excursions.

17. An automatic insulin delivery system comprising:
a wearable drug delivery device;
a wearable continuous blood glucose monitor in wireless communication with the drug delivery device; and
a personal diabetes management application, executing on the drug delivery device for performing the functions of:
receiving a quantity of a manually-administered basal dose of insulin;
determining, during the course of a period of time, that a blood glucose excursion has occurred based on one or more blood glucose readings from a continuous blood glucose monitor;
calculating a bolus dose of insulin to address the blood glucose excursion;
administering the bolus dose; and
based on the blood glucose excursion, recommending an adjustment to the quantity of the manually-administered basal dose for a subsequent period of time.

18. The automatic insulin delivery system of claim 17 wherein the personal diabetes management application performs the further functions of:
receiving a notification of a meal event, the notification comprising a macronutrient profile of the meal;
calculating a bolus dose of insulin based on the macronutrient profile to compensate for elevated blood glucose levels after the meal event; and
administering the bolus dose of insulin using the wearable drug delivery device.

19. The automatic insulin delivery system of claim 17 wherein the personal diabetes management application performs the further functions of:
determining a meal event based on one or more readings from the continuous glucose monitor indicating an elevated blood glucose level;
calculating a bolus dose of insulin based on the one or more readings; and
administering the bolus dose of insulin using the wearable drug delivery device.

20. The automatic insulin delivery system of claim 17 wherein:
a blood glucose excursion is determined to have occurred after receiving a predetermined number of consecutive blood glucose readings above a high threshold or a predetermined number of consecutive blood glucose readings below a low threshold; and
the adjustment to the manually-administered basal dose for the subsequent period of time is based on the number of consecutive blood glucose readings exceeding the high threshold or low threshold.
